(19) United States
(12) Patent Application Publication
(10) Pub. No.: US 2004/0110124 A1
(43) Pub. Date: Jun. 10, 2004

(54) Method of enhancing radiation resistance of normal cells

(76) Inventors: Susan Jane Knox, Stanford, CA (US); Shoucheng Ning, Palo Alto, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/680,959

(22) Filed: Oct. 7, 2003

Related U.S. Application Data
(60) Provisional application No. 60/417,012, filed on Oct. 7, 2002.

(51) Int. Cl. C12Q 1/00

(52) U.S. Cl. 435/4

(57) ABSTRACT

Methods are provided for increasing the resistance of normal cells to radiation damage, by administering an agent that induces hyperpolarization of the targeted cell membrane. Hyperpolarization may be induced prior to radiation exposure, or immediately afterwards, and preferably is continued for a period of time after exposure. Methods are also provided for means of screening candidate agents for use in radioprotection.

### BACKGROUND

It is well-known that radiation is damaging to cells. Initial deposition of energy in irradiated cells occurs in the form of ionized and excited atoms or molecules distributed at random throughout the cells. It is the ionizations that cause most of the chemical changes in the vicinity of the event, by producing a positively charged or “ionized” molecule. These molecules are highly unstable and rapidly undergo chemical change to produce free radicals, atoms, or molecules containing unpaired electrons. These free radicals are extremely reactive and can lead to permanent damage of the affected molecule.

As an immediate consequence of radiation damage, cells can undergo apoptosis, dying in interphase within a few hours of irradiation. Typical morphologic changes include loss of normal nuclear structure and degradation of DNA. Although DNA damage is thought to be important in triggering such an apoptotic response, some studies have suggested a role for membrane damage and signaling pathways outside the nucleus that involve tyrosine kinases, especially ceramide. It has been proposed that p53-dependent apoptosis may involve the transcriptional induction of redox-related genes with the formation of reactive oxygen species, leading to cell death by oxidative stress.

Another mechanism for cell killing is radiation-induced reproductive failure. Radiation in sufficient doses can inhibit mitosis. The inhibition of cellular proliferation is the mechanism by which radiation kills most cells. As radiation kills cells by inhibiting their ability to divide, its effects in human beings occur primarily in tissues with high cell turnover or renewal rates characterized by a large amount of proliferative activity. These include tissues such as the bone marrow and the mucosal lining of the stomach and small intestine.

Radiation can induce chromosomal aberrations, both lethal and non-lethal. These include such changes as dicentrics, ring chromosomes, large deletions, and fragments, which do not allow the equal distribution of genetic material into daughter cells; and changes such as small deletions, reciprocal translocations, and aneuploidy.

The investigation and development of effective radioprotectors is of great importance to populations potentially subjected to accidental, intentional or military exposure to ionizing radiation. Effort has been put into the development of chemical compounds with anti-oxidant properties that could potentially be taken prior to exposure to the radiation. However, current limitations of such chemical radioprotectors are that they are not long lasting, and there are toxicities associated with their use at cytoprotective doses. Therefore, effective and well tolerated protection of normal tissues from radiation injury will require novel strategies. The present invention addresses this need.

Relevant Literature

Association of Bcl-2 with membrane hyperpolarization and radioresistance is described in Gilbert et al. (1996) J. Cell. Physiol. 168:114-122. Resistance to apoptosis by depletion of cellular thiols is described in Mirkovic et al. (1997) Oncogene 15:1461-1470.

Agents that open potassium channels and result in membrane hyperpolarization have been described as potential therapeutic agents. Such diseases or conditions include asthma, epilepsy, hypertension, impotence, migraine, pain, urinary incontinence, stroke, Raynaud's Syndrome, eating disorders, functional bowel disorders, and neurodegeneration. See, for example, Lawson (2000) Kidney Int 57(3):838-45; McPherson (1993) Gen Pharmacol 24(2):275-81; Haeusler (1990) Clin Physiol Biochem8 Suppl 2:46-56; U.S. Pat. No. 6,265,417; among others.

### SUMMARY OF THE INVENTION

Methods are provided for increasing the resistance of normal cells to radiation damage, by administering an agent that induces hyperpolarization of the targeted cell membrane. Hyperpolarization may be induced prior to radiation exposure, or immediately afterwards, and preferably is continued for a period of time after exposure. Methods are also provided for means of screening candidate agents for use in radioprotection.

### BRIEF DESCRIPTION OF THE DRAWINGS

FIG. 1. Fluorescence measurement of Rat-1 cells before (Line A) and after (Line B) valinomycin (1.0 μM) treatment. The decrease in fluorescent reading (Line B compared to Line A) after valinomycin incubation for 2 hours indicates the hyperpolarization of the plasma membrane.

FIG. 2. Effect of valinomycin on radiation response of normal fibroblast cells. Rat-1 fibroblast cells were irradiated with 0-20 Gy in presence or absence of valinomycin. Valinomycin (0.01-1.0 μM) was added in the growth media immediately prior to irradiation, and removed at 2 hours or 3 days post irradiation. Three days after irradiation, cell survival was assessed by using the MTT assay.

FIG. 3. Effect of valinomycin on radiation response of normal endothelial cells. MS1 endothelial cells were irradiated with 0-20 Gy in the presence or absence of valinomycin. Valinomycin (0.01-1.0 μM) was added in the growth media immediately prior to irradiation, and removed at 2 hours or 3 days post irradiation. Three days after irradiation, cell survival was assessed by using the MTT assay.

FIG. 4. Effect of valinomycin on radiation response of normal epithelial cells. MDCK epithelial cells were irradiated with 0-20 Gy in the presence or absence of valinomycin. Valinomycin (0.01-1.0 μM) was added in the growth media immediately prior to irradiation, and removed at 2 hours or 3 days post irradiation. Three days after irradiation, cell survival was assessed by using the MTT assay.

### DETAILED DESCRIPTION OF THE EMBODIMENTS

Methods are provided for radioprotection of cells, by administering an agent that induces hyperpolarization of the cell membrane. Hyperpolarization may be induced prior to radiation exposure, or immediately afterwards for a radioprotective effect. Included among agents that increase membrane hyperpolarization are potassium channel openers.

Agents useful for radioprotection are capable of membrane hyperpolarization, for example by opening the potassium channel in a cell membrane. An effective dose will generally hyperpolarize the membrane sufficiently to prevent radiation-induced cell killing. The host, or patient, may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease.

Surprisingly, it is found that membrane hyperpolarization allows normal, i.e. non-transformed, cells to resist radiation damage. Types of radiation damage include chromosomal aberrations, induction of apoptosis, mitotic arrest, and the like. By the term “normal” it is intended to refer to cells that are not transformed, i.e. are not cancer cells. Normal cells are subject to growth control and regulation, for example cells that are normally present in a living organism, primary cell culture, and the like.

The methods of the invention are useful in prevention of radiation damage from a variety of sources of ionizing radiation, including X-rays, gamma-rays, beta radiation and alpha radiation. Such radiation may result from exposure to nuclear fusion or fission materials, e.g. nuclear waste, nuclear weapons, or nuclear power plants, from intentional or unintentional exposure to radiation, e.g. X-rays, gamma rays, etc. for medical or other purposes.

Therapeutic formulations of hyperpolarizing agents are provided. In one aspect of the invention, the hyperpolarizing agent is administered to individuals having an increased susceptibility for radiation toxicity. In adults, radiation toxicity may occur after a single dose of more than about 3 Gy. Children, or patients with such diseases as Fanconi anemia or Ataxia telangiectasia can have increased susceptibility for radiation toxicity. The formulations of the present invention find use as protective agents, for example in cancer patients treated with ionizing radiation. The hyperpolarizing agent can be administered locally (e.g. rectally) to protect the rectal mucosa during pelvic radiation therapy, or systemically to protect specific radiosensitive tissues, e.g. bone marrow, etc. against anticipated radiation exposure, e.g. radiation therapy or exposure resulting from workplace radiation, military exposure, and the like. In another embodiment, the hyperpolarizing agent is administered locally or systemically immediately following accidental or unintentional exposure.

Hyperpolarizing agents are administered to an animal that has been exposed, or that may be exposed to radiation. Administration may be topical, localized or systemic, depending on the specific exposure. The compounds of the present invention are administered at a dosage that protects the cell population while minimizing any side-effects. It is contemplated that the composition will be obtained and used under the guidance of a physician for in vivo use. The dosage of the therapeutic formulation will vary widely, depending upon the nature of the disease, the frequency of administration, the manner of administration, the clearance of the agent from the host, and the like.

The membrane potential, the voltage difference across a cell's plasma membrane, depends on the distribution of ionic charge. When there is an accumulation of unequal positive and negative charges on either side of a membrane, a difference of electrical potential (voltage) is set up between the two sides of the membrane. Charge is carried back and forth across the cell membrane by small inorganic ions. These can traverse the lipid bilayer only by passing through special ion channels or pumps. When the ion channels open, the charge distribution shifts and the membrane potential changes.

Ion channels are multi-subunit, membrane bound proteins. In humans, ion channels comprise extended protein families with hundreds, or perhaps thousands, of both closely related and highly divergent family members. These channel superfamilies can be broadly classified into groups, based upon the specific ion, the type of gating, and the number of transmembrane domain segments and pore segments in the mature protein. The functional protein is a multimer that comprises one or more types of alpha subunits, and which are frequently associated with auxiliary, β subunits.

Of interest for the present methods are compounds that act on the Na⁺/K⁺-ATPase pump, which is the primary ion transport system responsible for maintenance of intracellular Na⁺ and K⁺ ion concentrations, and is the main contributor to the resting membrane potential of the a cell. The ability of the pump to maintain a normal resting membrane potential is important for cell homeostasis. However, the pump is susceptible to inactivation by reactive oxygen species generated by radiation or chemical photolysis.

A large body of literature exists in the general area of ion channels. A review of the literature may be found in the series of books, “The Ion Channel Factsbook”, volumes 1-4, by Edward C. Conley and William J. Brammar, Academic Press. An overview is provided of: extracellular ligand-gated ion channels (ISBN: 0121844501), intracellular ligand-gated channels (ISBN: 012184451X), inward rectifier and intercellular channels (ISBN: 0121844528), and voltage gated channels (ISBN: 0121844536). Hille, B. (1992) “Ionic Channels of Excitable Membranes”, 2^{nd} Ed. Sunderland MA:Sinauer Associates, also reviews potassium channels.

### HYPERPOLARIZING AGENTS

Hyperpolarizing agents for use in the methods of the invention may be determined by the screening methods set forth below. Alternatively, known hyperpolarizing agents may be used. Agents of interest are able to hyperpolarize the membrane with acceptable toxicity profiles at the therapeutic dosage; and protect normal, non-transformed cells from radiation damage. For example, a therapeutic dose can protect at least about 50% of cells in a sample from death after radiation at a dose of not more than about 10 Gy; usually a therapeutic dose can protect at least about 75% of cells in a sample from death after radiation at a dose of not more than about 10 Gy; and preferably a therapeutic dose can protect at least about 90% of cells in a sample from death after a radiation dose of not more than about 10 Gy.

Compounds of interest for use in the methods of the invention are chemically diverse agents, including, for example without limitation, cromakalim, pinacidil, nicorandil, RP-52891, valinomycin, methylxanthines, and the like, which evoke K⁺ efflux through adenosine 5′-triphosphate (ATP)-sensitive K⁺ channels (KATP). KATP channel openers demonstrate heterogeneous pharmacology indicative of independent sites of action for the different agents. Other compounds of interest, e.g. BKCa channel openers, include, without limitation, NS004, fenamates, DHS-I, maxikdiol, etc. For examples and discussions of K+ channel openers, see, inter alia, Haeusler (1990) Clin Physiol Biochem 8 Suppl 2:46-56; McPherson (1993) Gen Pharmacol 24(2):275-81; Lawson (2000) Kidney Int 57(3):838-45; U.S. Pat. No. 6,265,417 “Potassium channel openers”; U.S. Pat. NO. 5,972,894, “Peptides having potassium channel opener activity”.

Screening methods generally involve conducting various types of assays to identify agents that induce hyperpolarization, or that protect normal cells from radiation damage. Lead compounds identified during these screens, or known hyperpolarizing agents, can serve as the basis for the synthesis of more active and/or less toxic analogs. Lead compounds and/or active analogs generated therefrom can be formulated into pharmaceutical compositions effective in radioprotection.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples. The effect on membrane polarization may be measured, and/or the protection of cells against radiation damage. The changes in response to the agent are measured, desirably normalized. The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

The effect of agents on membrane polarization can be investigated by looking at the calcium flux through channels. Cells are loaded with calcium indicator dyes such as Fluo-3 or Fura-2, which change their fluorescence properties on calcium binding. Activation of channel opening and the resultant Ca²⁺ entry is measured by changes in the fluorescence using a fluorometric plate reader or fluorescence microscopy. The effects of candidate agents can be assessed. For example, measurements may be done in presence/absence of candidate agents by using a FLEX station/Flipper or Ca2+ Imaging (see Renard, S. et al. Eur. J. Physicology 366:319-328 (1999)). The Molecular Devices FLEX station is a scanning fluorometer coupled with a fluid transfer system that allows the measurement of rapid, real time fluorescence changes in response to application of compounds.

Whole cell voltage clamp experiments may be performed to record currents passing through channels in the absence and presence of candidate agents. Patch clamp recording may be used to measure single channel currents from inside-out patches. The transmembrane potential of the cell may be modulated by the use of repetitive electrical stimulation to cycle the ion channel of interest through its activation cycle, and to set the transmembrane potential to a desired level suitable for a specific activation state, or transition between states. During or after this process a test compound is added to the cell, and the transmembrane potential is measured.

A number of methods are known in the art for recording the currents and electrical potential changes of cell membranes. These systems may utilize an intracellular or extracellular electrode, preferably an extracellular electrode, operably contacting the cell membrane; and a reference electrode outside of the cell. The electrodes are connected to an amplifying means and a recording means. Typically, the output signal is recorded as changes in voltage (or current) over time. This information can then be digitized for further analysis or analyzed via analog means.

### FORMULATIONS

The compounds of this invention can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

In pharmaceutical dosage forms, the compounds may be administered in the form of their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres; slabs, etc. with biodegradable or non-biodegradable polymers. For example, polymers of lactic acid and/or glycolic acid form an erodible polymer that is well-tolerated by the host. The implant containing the inhibitory compounds may be placed in proximity to the site of a tumor, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to, be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

MATERIALS AND METHODS

Three normal cell lines (Rat-1, a fibroblast cell line; MS-1, an endothelial cell line and MDCK, a epithelial cell line) were studied. All three cell lines were cultured in Dulbecco's modified Eagle medium supplemented with 10% fetal calf serum, 100 U/ml penicillin and 100 ug/ml streptomycin (Gibco BRL, Gaithersburg, Md.) at 37° C. with 5% CO₂. Cells were irradiated with a ¹³⁷Cs source at a dose rate of 300 cGy/min, and the viability of cells was measured by the MTT assay. Valinomycin was used as an agent to hyperpolarize the plasma membrane. The membrane potential was recorded with a fluorescent probe bis-oxonol, a negatively charged potential-sensitive fluorescent dye, which is distributed across the plasma membrane. Binding of this probe to intracellular components is dependent upon the membrane potential.

Results

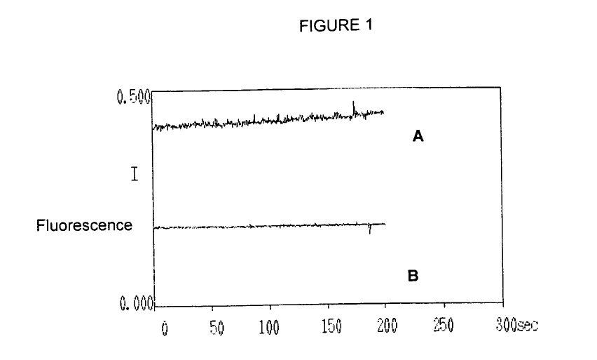

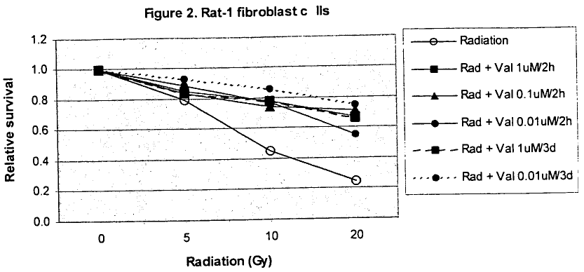

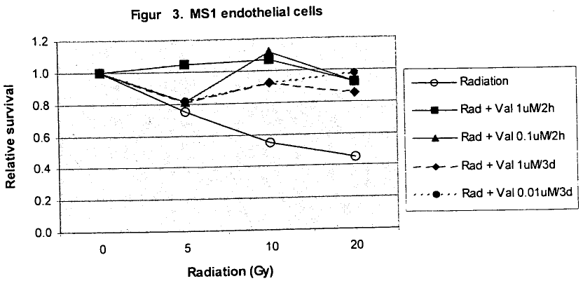

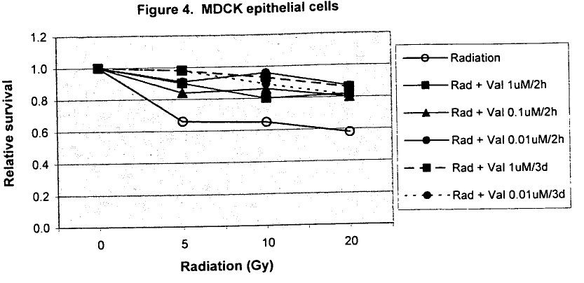

Treatment of cells with valinomycin at a concentration range of 0.01-1 nM for 2 hours or 3 days decreased the intracellular fluorescence by 55-60% (from 0.44-0.5 measured prior to valinomycin treatment to 0.18-0.20), indicating that valinomycin hyperpolarized the plasma membrane (FIG. 1). FIGS. 2-4 show the radiation responses of three cell lines treated with or without valinomycin. All three cell lines showed dose-dependent responses to radiation. The relative survivals (% of control) of Rat-1, MS-1 and MDCK cells irradiated at 20 Gy were 25%, 45% and 58%, respectively. Treatment of cells with 1.0 μM valinomycin for 2 hours increased the survival from 25% to 67% for Rat-1, from 45% to 92% for MS-1 and from 58% to 82% for MDCK cells, representing a 2.7-1.4-fold increase in the relative survival following irradiation. Valinomycin treatment with the concentrations of 0.01 μM to 1.0 μM produced very similar radiation protective effects in all three cell lines.

These data demonstrate that the membrane potential plays an important role in radiosensitivity of normal cells. Hyperpolarizing the cellular plasma membrane will protect normal cells from radiation damage.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

What is claimed is:

1. A method of protecting normal cells from radiation damage, the method comprising:
contacting said normal cells with a dose of a hyperpolarizing agent effective to hyperpolarize the cell membrane;
wherein said normal cells are protected from radiation damage.

2. The method according to claim 1, wherein said contacting is performed prior to radiation exposure.

3. The method according to claim 1, wherein said contacting is performed immediately after radiation exposure.

4. The method according to claim 1, wherein said radiation is ionizing radiation.

5. The method according to claim 4, wherein said ionizing radiation results from nuclear fission or fusion.

6. The method according to claim 4, wherein said ionizing radiation is X-rays.

7. The method according to claim 1, wherein said hyperpolarizing agent in a potassium channel opener.

8. The method according to claim 7, wherein said potassium channel is the KATP channel.

9. A method for identifying an agent as radioprotective agent, comprising:
combining a candidate agent with a cell;
measuring polarization of the cell membrane;
wherein an agent that hyperpolarizes said cell membrane, provides for protection against radiation damage in normal cells.

10. The method according to claim 9, wherein said changes are measured by calcium flux.

11. The method according to claim 9, wherein said changes are measured by patch clamping.
